(19) United States
(12) Patent Application Publication
(10) Pub. No.: US 2004/0005695 A1
(43) Pub. Date: Jan. 8, 2004

(54) Method for producing recombinant proteins by gram-negative bacteria

(76) Inventors: Gerhard Miksch, Steinhagen (DE); Erwin Flaschel, Biefeld (DE); Roland Breves, Ratingen (DE); Karl-Heinz Maurer, Erkrath (DE); sophia Kleist, Biefeld (DE)

Correspondence Address: Connoly Bove Lodge & Hutz, 1220 Market Street, P O Box 2207, Wilmington, DE, 19899, US

(21) Appl. No.: 10/258,367

(22) PCT Filed: Apr. 12, 2001
(86) PCT No.: PCT/EP01/04227

(30) Foreign Application Priority Data
Apr. 20, 2000 (DE) 100 19 881.3

(51) Int. Cl. C12N 1/12; C12N 1/20

(52) U.S. Cl. 435/252.1

(57) ABSTRACT

The invention relates to a method for producing recombinant proteins by gram-negative bacteria. According to the inventive method, the products are released into the surrounding medium, thereby allowing for high expression and production rates. To this end, the gene of the recombinant protein to be produced is placed under the control of a promoter derived from a gram-positive organism, preferably from a promoter derived from the genus Bacillus that in nature does not control said gene, and a system becomes active that partially opens the outer membrane of the bacteria produced. The preferred bacteria are E. coli or Klebsiella, promoters that are not necessarily inducible from outside, especially constitutive promoters such as the β-glucanase promoter of Bacillus amyloliquefaciens (bgl promoter) and the colicin system. The protein is thereby released into the surrounding medium from where it can be easily purified. The inventive method allows for making the fermentative production of protein more efficient. The inventive system is for example suitable for producing α-amylases or bacterial phytases.

The present invention relates to a method for producing recombinant proteins by Gram-negative bacteria, in particular E. coli or Klebsiella. Said method is distinguished in that the products are secreted into the surrounding medium and that it is possible in this way to obtain high expression and production rates. This is achieved by creating the gene of the recombinant protein to be produced under the control of a promoter from a Gram-positive organism, preferably from an organism of the genus Bacillus, which does not naturally regulate said gene and by a system becoming active which partially opens the outer membrane of the producing bacteria.

Gram-negative bacteria, in particular Escherichia coli and Klebsiella, are frequently used in genetics. In contrast, only in a few cases are Gram-negative organisms used for industrial enzyme production. There, instead, advantage is taken of the fact that in particular Gram-positive bacterial species such as Bacillus or Arthrobacter or fungi such as Aspergillus or Trichoderma naturally secrete hydrolytic enzymes such as cellulases, amylases, proteases or pectinases. It is therefore possible to obtain these enzymes readily and efficiently from the particular culture medium for these microorganisms. Yeasts such as Saccharomyces or Kluyveromyces are likewise utilized for protein production, owing to their own enzymes, but also because they can be managed genetically and micro-biologically in a simple manner similarly to bacteria and because they are, as eukaryotes, capable of the appropriate posttranslational modifications of the proteins.

Gram-negative bacteria can be used in principle for producing eukaryotic proteins such as, for example, insulin via methods of genetic engineering known per se. However, a fundamental problem here is the fact that the transgenically obtained proteins, often after correct transcription and translation, are present inside the cell as aggregates (“inclusion bodies”); or they are, if they have the appropriate N-terminal signal sequence which can be recognized and cleaved off by the bacterium, transported through the inner membrane into the periplasm but not through the outer membrane into the surrounding culture medium as well. Therefore, conventional purification of the particular product from Gram-negative bacteria requires cell disruption or lysis of the outer membrane and is thus comparatively complicated and expensive.

A satisfactory solution to this problem would provide Gram-negative bacteria for a broad new field of application, namely the industrial production of proteins, and appears to be particularly advantageous, especially because of the genetic knowledge about these organisms, and an economical alternative, due to their short generation times in comparison with eukaryotic cells.

Moreover, exporting the proteins produced into the medium surrounding the cells would also, compared to periplasmic localization, facilitate the formation of disulfide bridges and thus correct folding of the proteins (Biotechnology (1991), Vol. 9, pp. 545-551; Gene (1992), Vol. 116, pp. 129-138) and thus provide said proteins with better protection from being degraded again by the producing cells (Methods Enzymol. (1990), Vol. 185, pp. 166-187; Kresze, G. D., in: Seetharan, S., Sharma, S. K. (Editors), Purification and analysis of recombinant proteins; Dekker, New York, pp. 85-120).

An example of economically important proteins whose production processes are in urgent need of improvement are phytases. These enzymes (E.C. 3.1.3.26) are important in animal breeding. They have previously been obtained by culturing those fungi which produce them naturally, for example Aspergillus niger. These fungi, however, require economically disadvantageous culturing conditions, for example because they have generation times of up to 100 h. The study by Greiner, R. Konietzky, U., Jany, K. -D. from 1993 in Arch. Biochem. Biophys., Vol. 303, pp. 107-113 describes, for the first time, bacterial phytases, namely from the Gram-negative bacterium Escherichia coli. According to this, E. coli phytase has an activity which is many times higher than that of the known fungal phytases. Moreover, the E. coli generation time is approx. 20% of that of the abovementioned fungi. However, the fermentation of E. coli is accompanied by the above-described problems. Using E. coli for the production of these E. coli-native enzymes would make the production of these economically important enzymes considerably more efficient.

BRP (bacteriocin release protein) has already been used as a system for partially opening the outer membrane of Gram-negative bacteria. It acts by exporting heterologously expressed proteins from the periplasm of the producing Gram-negative bacteria into the surrounding medium (compare references in Arch. Microbiol. (1997), Vol. 167; pp. 143-150). This system, however, has a disadvantage in that it may lead to a cell lysis which is too severe or reduce the viability of the cells.

A further membrane-opening system is the colicin system found in some Gram-negative bacteria such as Escherichia coli. These possess naturally the lysis or Kil gene (J. Bacteriol. (1983), Vol. 153, pp. 1479-1485) whose activity causes the cells to die. The property of the Kil protein to lyse the outer membrane of Gram-negative bacteria was employed in the European patent application EP 335567. This property makes it possible for recombinant proteins which are produced by the Gram-negative bacterium and, according to the known prior art, transported into the periplasm with the aid of the appropriate signal sequence to move from the periplasm into the surrounding nutrient medium. The activity of the kil gene itself is crucial in this kind of system since it leads, if too high, to a complete cell lysis, as in a stationary bacterial culture (J. Bacteriol. (1986), Vol. 168, pp. 648-654). Thus, in the patent application cited it is placed under the control of strong inducible promoters such as those for lacZ, trp or lambda-P_{L}, thereby achieving a controlled release. In the particular application, expression of the transgene is not regulated individually but takes place via the same promoters as those for controlling the kil gene. In contrast to this, continuous protein production accompanying the bacterial growth and/or a release into the medium, which is sufficient for production, would be desirable.

The study “Extracellular production of a hybrid β-Glucanase from Bacillus by Escherichia coli under different cultivation conditions in shaking cultures and bioreactors” (G. Miksch, R. Neitzel, E. Fiedler, K. Friehs and E. Flaschel (1997), Appl. Microbiol. Biotechnol., Vol. 47, pp. 120-126) uses stationary phase-induced E. coli promoters (fic and bol A) for controlling expression of the kil gene. The host organism was Escherichia coli. In these experiments, β-glucanase which was used as indicator enzyme was produced constitutively under the control of its own promoter and, owing to its enzymic activity, could be detected in the supernatant. This study was intended to resolve the question as to whether expression was at all possible, for which question a gene under the control of its own promoter may be a suitable indicator. The strength of expression achievable and/or the amount of protein produced were not of interest in this study. Thus, it was not yet possible to consider a use of precisely this promoter for expressing another gene, in particular for the industrial production thereof.

In fact, this study concerned a hybrid glucanase, i.e. an enzyme composed in equal parts of the two β-glucanases from Bacillus macerans and B. amyloliquefaciens (Borriss et al., Carlsberg. Res. Commun., Vol. 54 (1989), pp. 41-54); the N-terminal half was that of Bacillus amyloliquefaciens β-glucanase and the C-terminal half that of B. macerans β-glucanase. These two proteins are 70% identical at the amino acid level. Thus, in the study mentioned the gene in question has, at least partially, been under the control of its own promoter; in particular, the transition of the promoter region to the protein-coding part was identical to the in vivo situation. These enzymes must at least be regarded as being highly homologous.

In Klebsiella planticola (Appl. Microbiol. Biotechnol. (1999), 51; 627-632), the corresponding experiment using the fic promoter for the kil gene and the bgl promoter for the same detection enzyme was likewise successful. Here too, the transgene was thus again under the control of a promoter which naturally regulates partly the same or at least a highly homologous protein. Here too, it was possible to increase both product formation and product secretion compared to the non-kil-expressing control.

In the case of the application DE 19823216, too, the same indicator enzyme has been expressed in K. planticola, and again under the control of the promoter of Bacillus amyloliquefaciens β-glucanase. Secretion was made possible by the kil system.

G. Miksch, E. Fiedler, P. Dobrowolski and K. Friehs again investigated in the publication “The kil gene of the ColE1 plasmid of Escherichia coli controlled by a growth-phase-dependent promoter mediates the secretion of a heterologous periplasmic protein during the stationary phase” (Arch. Microbiol. (1997), Vol. 167, pp. 143-150) the heterologous protein expression in E. coli. This study showed that the promoter of the E. coli fic gene (filamentation induced by cAMP) is suitable for regulating expression of the Kil protein if, at the same time, the indicator enzyme is constitutively produced under the control of its own natural promoter. Here too, the hybrid glucanase thus served again as indicator enzyme. As a result, product formation is increased compared to the control without kil activity but with the same constitutive production of the indicator enzyme.

For heterologous protein expression by the host organism Acetobacter methanolicus (Appl. Microbiol. Biotechnol. (1997), Vol. 47, 530-536), both the kil gene and the transgene (again the hybrid glucanase) were regulated by the stationary phase-specific fic promoter.

The aim of all these experiments was to establish the colicin system in the various host bacteria and, respectively, find suitable conditions under which transgene can be produced and exported, without the cells dying due to the activity of the Kil protein. In this connection, secretion caused an increase in the expression, i.e. an increase in the product formation rate. In the case of Klebsiella, exporting the protein produced actually caused its overexpression in the first place, since usually this organism is not suitable for heterologous protein expression. However, in order to control the transgene which had been derived from the genes of Gram-positive organisms, promoters which naturally regulate part of this gene or a highly homologous gene were used in each of these cases. Owing to these results, it cannot readily be assumed that, in a comparable context, an expression in which the protein to be produced is under the control of a promoter which naturally regulates neither this gene nor any highly homologous gene but rather a completely different gene can also be successful.

The gene of Bacillus amyloliquefaciens β-glucanase is a common indicator for the activity of other promoters. The use of the β-glucanase promoter (bgl promoter) itself for controlled expression of recombinant proteins, however, is not common, in particular not in the case of proteins which are not naturally regulated by the promoter itself or which are not highly homologous to these proteins (see above; compare Borriss et al., Carlsberg. Res. Commun., Vol. 54 (1989), pp. 41-54). This promoter is constitutive, i.e. it need not be specifically activated by being acted upon from the outside. In contrast, promoters to be specifically activated have been used for heterologous protein expression in the prior art up until now. Examples of these are the P_{lacz} and Pₜᵣₚ promoters which can be induced by the addition of appropriate chemicals and the P_{L} promoter of the bacterial phage lambda, which can be induced by an increase in temperature (EP 335567).

Against this background, it is the object of the present application to establish a system according to which recombinant proteins can be obtained in high yield from the culture supernatant during or after fermentation of Gram-negative bacteria. Part of the object was to find a system which partially opens the outer membrane of the Gram-negative bacteria, without the majority of the producing bacteria being lysed completely and dying.

Another object of the present invention was to find a promoter for regulating heterologous genes, which is as powerful as possible in the presence of a functioning colicin system. Particularly advantageous for efficient production would be the use of a promoter which need not necessarily be induced from the outside during the course of production.

According to the invention, these objects are achieved by those methods for producing recombinant proteins by Gram-negative bacteria according to which the proteins are secreted at least partially into the medium surrounding the bacteria with the aid of a system which partially opens the outer membrane of said bacteria and which are furthermore characterized in that the recombinant protein to be produced is expressed under the control of a promoter from a Gram-positive organism, preferably from an organism of the genus Bacillus, which promoter does not naturally regulate the corresponding gene or a gene highly homologous to this gene.

As a result, Gram-negative bacteria are also made available for industrial protein production and thus alternative production systems are added to the prior art. These alternative production systems are particularly advantageous because a comprehensive wealth of knowledge with respect to their genetics, their microbiology and their biotechnological potential is available for them on the laboratory scale.

For example, using Gram-negative organisms produces shorter generation times compared to fungi, resulting in production which is overall more cost-effective. Another advantage of the present invention is the possibility of proteins from Gram-negative organisms being produced by these organisms themselves on an industrial scale. To this end, it is no longer necessary to switch to Gram-positive or other expression systems. Thus, the optimal conditions generated for this by evolution are utilized, for example with respect to the transcription and translation apparatus or codon usage.

The present invention relates firstly to a method for producing a recombinant protein by Gram-negative bacteria, which protein is secreted at least partially into the medium surrounding said bacteria with the aid of a system which partially opens the outer membrane of said bacteria, which method is characterized in that the recombinant protein to be produced is expressed under the control of a promoter from a Gram-positive organism, preferably from an organism of the genus Bacillus, which promoter does not naturally regulate the corresponding gene or a gene highly homologous to this gene.

Embodiments of this subject matter of the invention are appropriate methods which are characterized in that the Gram-negative bacteria are coliform bacteria, in particular those of the genera Escherichia coli and Klebsiella; in that the coliform bacteria are derivatives of Escherichia coli K12, of Escherichia coli B or Klebsiella planticola, very particularly those of the strains Escherichia coli BL21 (DE3), E. coli RV308, E. coli DH5α, E. coli JM109, E. coli XL-1 and Klebsiella planticola (Rf); and/or in that the microorganism is the strain deposited with the application number DSM 14225 or a derivative of this strain.

Further embodiments of this subject matter of the invention are appropriate methods which are characterized in that the system which partially opens the outer membrane is the E. coli colicin system, in particular the Kil protein and/or a system under the control of the fic promoter or of another stationary-phase promoter.

Further embodiments of this subject matter of the invention are appropriate methods which are characterized in that the expression promoter is a promoter which need not necessarily be induced from the outside, preferably a constitutive promoter and particularly preferably the Bacillus amyloliquefaciens β-glucanase promoter.

Further embodiments of this subject matter of the invention are appropriate methods which are characterized in that secretion competence is mediated via a secretion cassette, in particular one which has been integrated into the chromosome; in that the expression cassette and the secretion cassette are located on different replicons; in that the expression cassette is located on the same replicon as the secretion cassette, in particular in the form of the expression cassette being located immediately upstream or downstream of the secretion cassette; and/or in that the expression cassette and the secretion cassette are located on an autonomously replicating plasmid which can replicate autonomously, preferably on the same plasmid.

Further embodiments of this subject matter of the invention are appropriate methods which are characterized in that the protein is an enzyme which is in particular a hydrolase, in particular an amylase, glucanase, protease, lipase or cellulase; in that the recombinant proteins produced are phytases, in particular bacterial phytases; in that the phytase is secreted by using the E. coli kil gene under the control of an E. coli stationary-phase promoter, preferably the fic promoter; in that the membrane-opening system, in particular the kil gene, is provided via a secretion cassette; in that the gene of the phytase is under the control of the Bacillus amyloliquefaciens β-glucanase promoter; in that the host strain used is Escherichia coli BL21 (DE3); and/or in that the expression vectors used are the vectors pPhyt109 or pPhyt119/4 or vectors derived therefrom.

The second subject matter of the invention are secretion cassettes which possess the genetic elements responsible for the membrane-opening properties of the membrane-opening system, in particular the E. coli colicin system and/or a stationary-phase promoter, very particularly the gene for the Kil protein and/or an E. coli stationary-phase promoter including, in particular, the fic promoter.

Further embodiments of this subject matter of the invention are appropriate secretion cassettes which are characterized in that they additionally contain an expression cassette located immediately upstream or downstream, which contain the transgene and a promoter as its control element, which is in particular a promoter which need not necessarily be induced from the outside, preferably a constitutive promoter and particularly preferably the Bacillus amyloliquefaciens β-glucanase promoter; and/or in that they contain as transgene the gene for an enzyme, preferably that of a hydrolase, in particular that of an amylase, glucanase, protease, lipase or cellulase, or that of a bacterial phytase.

The third subject matter of the invention are vectors which can replicate in Gram-negative bacteria and which contain a secretion cassette according to the second subject matter of the invention, in particular those which additionally contain the expression cassette. Further embodiments of this subject matter of the invention are appropriate expression vectors for Gram-negative bacteria, in particular for coliform bacteria, among these in particular for those of the species Escherichia coli or Klebsiella, very particularly any of the vectors pAmy63, pPhyt 109 and pPhyt119/4 or those which can be derived from any of these vectors, in particular by replacing the gene to be expressed; and/or cloning vectors containing a secretion cassette according to the second subject matter of the invention.

The fourth subject matter of the invention are Gram-negative bacterial strains which carry in a vectorial location a secretion cassette according to the second subject matter of the invention, in particular coliform bacterial strains, very particularly of the genera Escherichia coli and Klebsiella, and among these in particular derivatives of E. coli K12, E. coli B or Klebsiella platicola. Among these, those which are derived from E. coli BL21 (DE3), E. coli RV308, E. coli DH5α, E. coli JM109, E. coli XL-1 or from Klebsiella platicola (Rf) or from the strain deposited with the application number DSM 14225 are in turn preferred.

Further embodiments of this subject matter of the invention are appropriate bacterial strains which are characterized in that they contain an expression vector with a promoter and a gene regulated by said promoter; and/or in that they have been obtained after transformation with any of the vectors according to the third subject matter of the invention.

This subject matter of the invention also includes all bacterial strains which are characterized in that they carry in a chromosomal location a secretion cassette according to the second subject matter of the invention, in particular coliform bacteria, and among these in particular strains of Escherichia coli or Klebsiella, preferably of derivatives of Escherichia coli K12 or Escherichia coli B or Klebsiella planticola, very particularly of those of the strains Escherichia coli BL21 (DE3), E. coli RV308, E. coli DH5α, E. coli JM109, E. coli XL-1 or Klebsiella planticola (Rf) and among these in particular of those of the strain deposited with the application number DSM 14225; and/or in that they express the recombinant protein under the control of a promoter which need not necessarily be induced from the outside, preferably of a constitutive promoter and particularly preferably of the Bacillus amyloliquefaciens β-glucanase promoter (bgl promoter). Among these, particular preference is given to derivatives of the microorganism deposited with the application number DSM 14225. Another embodiment of this subject matter of the invention are microorganisms which are characterized in that they have been obtained after transformation with any of the vectors according to the third subject matter of the invention.

The fifth subject matter of the invention are methods for fermentation of Gram-negative bacteria producing a recombinant protein which is at least partially secreted into the medium surrounding said bacteria with the aid of a system which partially opens the outer membrane of said bacteria, which methods are characterized in that the recombinant protein is expressed under the control of a promoter from a Gram-positive organism, preferably from an organism of the genus Bacillus, which promoter does not naturally regulate the corresponding gene or a gene highly homologous to this gene. This subject matter of the invention includes appropriate methods which are characterized in that bacteria according to the fourth subject matter of the invention are used; in that the fermentation is carried out via a continuous supply strategy; in that the protein produced is subsequently harvested from the fermentation medium; and/or in that the protein produced is removed continuously during the fermentation.

The examples of the present application illustrate the manner in which the subject matters of the invention, in particular methods of the invention, can be realized. They especially elucidate the construction of appropriate secretion strains in which the responsible genes may be located on a plasmid or chromosomally. On the basis of this information, each example can in principle be reproduced. When generating a bacterial strain in which the relevant genetic elements are located chromosomally, however, it is not possible to predict into which position on the chromosome the relevant elements will recombine. It is possible that essential genes may thereby be impaired and thus recombinants may be obtained which are viable only with difficulty, if at all. For this reason, a bacterial strain which had been successfully recombined according to said examples was deposited with a strain collection.

According to example 3 of the present application and according to Appl. Microbiol. Biotechnol. (1999), Vol. 51, pp. 627-632, it was possible to obtain a Klebsiella strain with chromosomal location of the secretion cassette, namely Klebsiella planticola (Rf)-FIC3/19. This strain is distinguished in that it carries, in the form of a chromosomal integration, the transposon Tn5-FIC3 which can be used for secretion according to the invention.

Said strain was deposited with the Deutsche Sammlung von Mikroorganismen und Zellkulturen GmbH, Brunswick, Germany on Apr. 9, 2001, following the rules of the Budapest Agreement from Apr. 28, 1977. The deposit number is DSM 14225.

It is the aim of the present application to improve the production of economically interesting proteins as recombinant proteins by Gram-negative bacteria.

Recombinant proteins in accordance with the present invention can mean both heterologously and homologously expressed proteins; in the former case, proteins are produced which are not naturally produced by the host bacterium employed as producer strain; in the latter case, those proteins which originate from the host bacterium itself are produced.

In accordance with the present application, a gene coding for a recombinant protein to be produced according to the invention is referred to as a transgene, despite the fact that, strictly speaking, each of the genetic elements introduced into the host cells is a transgene.

In principle, the present invention refers to all kinds of proteins which, however, must contain an N-terminal signal sequence which ensures periplasmic localization during the course of a normal bacterial protein synthesis. This localization is a requirement for the recombinant proteins to be able to be secreted according to the invention.

According to the invention, methods for producing recombinant proteins mean all genetic or microbiological methods which are based on the genes for the proteins of interest being introduced into a host organism suitable for production and being transcribed and translated by said host organism. The genes in question are suitably imported via vectors, in particular expression vectors. However, they may also be imported via those vectors which enable the gene of interest to be inserted into a genetic element already present in the host organism, such as the chromosome or other vectors. According to the invention, the functional unit of gene and promoter and possible further genetic elements is referred to as expression cassette; for this, however, it need not necessarily also be a physical unit.

The microorganisms suitable for production are cultured and fermented in a manner known per se, for example in batch systems or in continuous systems. In the former case, a suitable nutrient medium is inoculated with the recombinant bacterial strains and the product is harvested from the medium after a period which is to be determined experimentally. Continuous fermentations are distinguished by reaching a dynamic equilibrium in which, over a comparatively long period, cells partially die but also grow again and, at the same time, product can be removed from the medium.

A system which partially opens the outer membrane of the Gram-negative bacteria selected as host cells enables the proteins produced, in particular those produced recombinantly, to escape at least partially from the host bacteria into the surrounding medium.

The release of proteins into the medium surrounding the bacteria is in accordance with the present invention referred to as secretion, despite the biochemical mechanism on which this escape is based. Thus this term is not limited to processes which are, in connection with protein synthesis, naturally referred to as translocation through the particular membranes. Systems used according to the invention which partially open the outer membrane of Gram-negative bacteria add to a bacterial expression system the ability to export the products unspecifically, i.e. in a manner not based on the identity of the proteins, into the medium surrounding these bacteria cells.

However, the factors effecting this must not be so active as for the cells to be lysed completely and a majority of them to die. Examples of this are BRP (bacteriocin release protein; in Arch. Microbiol. (1997), Vol. 167: pp. 143-150), or the Kil protein known as part of the colicin system (J. Bacteriol. (1986), Vol. 168, pp. 648-654).

The functional unit mediating secretion competence, which need not necessarily also be a unit physically, is referred to as secretion cassette. Protein production according to the invention is then possible if the expression function and secretion competence are present in the same bacteria cell and are active at the same time, i.e. if the production strain in question combines the two genetic properties expression and secretion.

The proteins of interest can be obtained from the surrounding medium during or after fermentation in a manner known per se and in a less complicated way than if the product had to be purified from bacterial cytoplasm or periplasm. Possible techniques for purifying the protein from the medium are, for example, filtration, centrifugation, ammonium sulfate precipitation, gel chromatography, ion exchange chromatography and affinity chromatography.

Besides the easy obtainability, a further advantage of the present invention is the fact that the protein, as a result of its escaping over a long period, is constantly removed from the protein-synthesizing apparatus of the cell and therefore does not accumulate inside the cell. It may be assumed, independently of this theory, that the synthesis apparatus is thereby kept far from a chemical equilibrium so that production continues over a relatively long period and a high yield is achieved overall.

The use of promoters from Gram-positive organisms, preferably from an organism of the genus Bacillus, ensures initiation of protein synthesis and, advantageously, rates of expression which are higher than the rate of expression of the gene of interest under the control of its own constitutive promoter. Increasingly, preference is given to expression promoters which can achieve increasingly higher rates of expression. This positive effect is additionally enhanced by the controlled escape of the product formed into the surrounding medium. In each case it must be determined experimentally which promoters are suitable in the individual case. Variations of this kind can be understood on the basis of the procedure in example 1 of the present application. Surprisingly, it has been found that promoters from Gram-positive bacteria are particularly suitable for this.

The present invention is realized by using promoters which have the additional property of not naturally regulating the transgene or a gene highly homologous to this transgene, since this, together with the partially membrane-opening system, surprisingly seems to make possible a particularly good rate of production. Thus the present invention relates to expression cassettes containing promoters from Gram-positive organisms and transgenes which are less than 70% and increasingly preferably less than 65%, 60%, 55%, 50%, 45%, 40%, 35%, 30%, 25% and 20% identical at the amino acid level to the genes naturally regulated by said promoters. This applies in particular to the N-terminal regions of the genes in question and to the transitional region between the promoter and the start codon.

The method of the invention relates to Gram-negative bacteria since these contain a periplasm. Precisely these bacteria had the problem of recombinant proteins being secreted only insufficiently and these organisms thus being available only insufficiently for industrial protein production.

Owing to the comprehensive knowledge about coliform bacteria, for example with respect to molecular biological methods and culturability, said bacteria are preferred embodiments of the present invention. Particular preference is given to those of the genera Escherichia coli and Klebsiella, in particular nonpathogenic strains suitable for biotechnological production. The method of the invention is demonstrated in the examples of the present application using representatives of these genera.

Representatives of these genera are the K12 derivatives and the B strains of Escherichia coli and the species Klebsiella planticola. Strains which can be derived from these according to genetic and/or microbiological methods known per se and which can therefore be regarded as derivatives therefrom are very important for genetic and microbiological studies and are preferably used for developing methods of the invention. Such derivatives may be modified with respect to their demands on culturing conditions, for example via deletion or insertion mutagenesis, may have other or additional selection markers or express other or additional proteins. They may be in particular those derivatives which express, in addition to the protein produced according to the invention, further economically interesting proteins.

A multiplicity of K12 derivatives are available, for example E. coli XL-1 blue, E. coli JM109 (both from Stratagene, La Jolla, USA) or E. coli DH5α (ClonTech, Palo Alto, USA). Of the B strains, particular mention must be made of the strain E. coli BL21 (DE3) (Stratagene, La Jolla, USA; and Amersham Pharmacia Biotech, Freiburg, Germany). Owing to the ion mutation, this strain produces no extracellular proteases and carries the element DE3 integrated chromosomally as a requirement for the functioning of a T7 promoter possibly cloned into said strain which is used in the prior art for a multiplicity of clonings.

Further preferred starting strains for derivatizations according to the invention are the strains E. coli RV308, E. coli DH5α, E. coli JM109, E. coli XL-1 and K. planticola (Rf).

The strain E. coli RV308 (ATCC 31608) tested in the Hans-Knöll-Institut für Naturstoff-Forschung in Jena, Germany is described in J. Mol. Biol., Vol. 139 (1980), pp. 147-161. It is distinguished by not producing acetate (Appl. Microbiol. Biotechnol., Vol. 46 (1996), pp. 524-532) which may impair bacterial growth during fermentation.

Klebsiella planticola (Rf) is a rifamycin-resistant strain derived from Klebsiella planticola by spontaneous mutation (Appl. Microbiol. Biotechnol., Vol. 51 (1999), pp. 627-632). For molecular biological work and fermentation, this results in the advantage of it being possible to use this antibiotic for selection or for protecting the culture from infections.

The strains mentioned are frequently used in genetics and microbiology and are sold by commercial suppliers (see above). They are thus particularly important as starting points for the development of further bacterial strains of the invention. In a preferred embodiment, the system which partially opens the outer membrane is the E. coli colicin system, in particular the Kil protein.

Colicins are the polypeptides with bacteriocin-like action which are synthesized by particular pathogenic strains of coliform bacteria. They are usually encoded by plasmids (the “Col factors”) but can be transferred to other bacteria via bacterial conjugation only by the activity of “transfer or mobility genes (mob)” (type I Col factors). Type II Col factors normally carry transfer genes themselves, i.e. they can be transferred with the aid of the gene products encoded by themselves. Col factors can integrate into the bacterial chromosome. The genes responsible for the properties of Col factors include in the same operon, in addition to those for colicin itself (cea) and the gene responsible for immunity, also the lysis or kil gene (J. Bacteriol. (1983), Vol. 153. pp. 1479-1485). This gene codes for a small lipoprotein which activates membrane-bound phospholipases (phospholipase A2) and renders the membrane permeable for colicins and thus, in the end, causes lysis of the membrane (EMBO J., Vol. 3 (1984), pp. 2393-2397).

The Kil protein which causes a release of product or of cell constituents and/or the corresponding gene or another element with identical action, known from the interaction with colicins, are together referred to in the present invention as colicin system. They make possible a secretion in accordance with the present invention, i.e. they partially open the outer membrane of Gram-negative bacteria and add to bacterial expression systems the ability to export the products unspecifically, i.e. in a manner not based on the identity of the proteins.

According to a further embodiment, another membrane-opening system, for example another BRP (bacteriocin release protein) under its own promoter, is effectively or preferentially placed under the control of the fic promoter.

In the method of the invention, the export-effecting Kil protein is under the control of a promoter which need not be induced by intervention from the outside, preferably under the control of its own natural promoter (fic promoter) and/or a promoter from the organism used for production. Surprisingly, the rate of lysis of the transgenic bacteria cells is so low here that only a part of the cells lyses completely and dies. The other part, however, continues to live, produces the protein of interest and exports said protein via the pores generated by the Kil protein into the surrounding nutrient medium.

Another possibility is to place the Kil protein, another BRP or another membrane-opening system under the control of another stationary-phase promoter which may be weaker or stronger than the fic promoter or may be activated slightly earlier or later or under different environmental conditions. This makes possible fine tuning with respect to the sensitive equilibrium between cell lysis and release of the desired proteins.

Preferred embodiments are characterized by promoters for expression of the protein to be produced, which need not necessarily be induced from the outside. Inducible means in this connection: switching on or off specifically from the outside, for example during a fermentation in progress; this is carried out by specific human intervention, for example by adding chemicals or by changing the incubation conditions such as, for example, the temperature (compare EP 335567). For the present invention, preference is given among the promoters not necessarily to be induced from the outside to constitutive promoters. These are regulated by the bacteria themselves during their growth and/or during fermentation. In particular, they need not be activated at a particular time by a specific intervention from the outside, and this makes it substantially easier to carry out the fermentative production. This, however, does not rule out the possibility of specifically inducing this promoter nevertheless by a human intervention beyond its natural regulation; instead, this possibility represents another embodiment of the present invention. Very particular preference is given to the Bacillus amyloliquefaciens β-glucanase promoter (bgl promoter).

In individual cases, known promoters may be assayed for their possible use in the methods of the invention and their product formation. Assay series of this kind are in principle familiar to the skilled worker. Such promoters can be amplified from chromosomal or plasmid DNA via molecular biological methods such as, for example, PCR and be inserted into vectors known per se. Their activity can be determined by the vector in question carrying, depending on said promoter, the desired transgene or an indicator gene, whose activity can be quantified. This procedure is described in example 1 of the present application.

A secretion cassette means according to the invention a genetic element which imparts the capability for secretion according to the invention. Thus, it contains at least the gene for the factor(s) which constitute(s) the membrane-opening system, suitably under the control of a promoter which, in this case, may be a stationary-phase promoter, for example. Advantageously, the secretion cassette additionally contains a selection marker, for example an antibiotic resistance, and border sequences such as uncommon restriction sites or transposon-derived inverted repeats, in order to facilitate excision and recombination of the secretion cassette.

Preference is given to embodiments of the present invention in which secretion competence is imparted via such a secretion cassette, because the latter can be genetically manipulated as a separate element, for example on cloning vectors, and be transferred into various host cells.

Preference is given to those embodiments in which the secretion cassette has been integrated into the chromosome, since secretion-competent strains of this kind can be used for the production of various proteins or for expression-promoter studies in that they need to be transformed just with the particular expression vector. Their secretion competence is already provided by the chromosome. An example of this is the strain Klebsiella planticola (Rf)-FIC/19 described in example 3 of the present application.

In the latter case, the expression cassette composed of promoter and transgene and the secretion cassette are located on different replicons. This enables flexible operation, for example when switching the production system to different target proteins, by replacing only the expression cassette or inserting into said expression cassette a different and/or a further gene and/or a different promoter. Similarly, it may also be desirable to introduce modifications to the secretion cassette, for example for fine-tuning the time or the extent of opening of the outer membrane.

Particular preference is given to methods which are characterized in that the expression cassette is located on the same replicon as the secretion cassette. This applies both to the chromosomal and the plasmid location. In the case of chromosomal location, an integration which is stable over many generations can be assumed in principle. The plasmid location makes possible a variation, in particular an increase in the copy number of the cassettes in question, and thus can effect a high yield. In both cases, both elements have the same copy number and can be genetically manipulated together, for example be excised and transferred to another genetic element.

Preferably, this coupling takes place in the form of the expression cassette being located immediately upstream or downstream of the secretion cassette. A cassette of this kind is -used in example 1 for the vector pAmy63 and in examples 2 and 3. The construction of this secretion cassette is described in Arch. Microbiol. (1997), Vol. 167, pp. 143-150). It contains the following elements: kanamycin-resistance gene (Km), kil gene (kil), fic promoter (P_{fic}), multiple cloning site and, as terminator, an omega interposon (Ω-cm; according to Prentki, P., Frisch, H. M. (1984), Gene, Vol. 29, pp. 303-313). It thus enables, via the fic promoter, a stationary phase-dependent activation of the kil gene product. It provides a multiple cloning site for integration of the gene of interest and of a promoter responsible for this gene. Integration of the transgene of interest and of the corresponding promoter, preferably immediately upstream or downstream of said elements, converts this actual secretion cassette to the “complete” secretion cassette or combined expression-secretion cassette.

In a particularly preferred embodiment, the expression cassette and the secretion cassette are located on a plasmid which can replicate autonomously in bacteria. Thus, this is a plasmid which has the appropriate genetic elements in order to be recognized by the DNA synthesis apparatus of the bacteria and can be passed on to the daughter cells. The expression and secretion cassettes are preferably located on the same plasmid so that in each case both can be passed on and kept at a fixed number ratio to one another. This makes it also possible for them to be transferred together to other producer strains.

According to the method of the invention, it is possible to produce any oligo- or polypeptides, proteins or enzymes, the only requirement being that they can be manipulated molecular-biologically, i.e. their genes can be cloned according to methods known per se and be transformed into host bacteria and be transcribed and translated there. The corresponding genes can be obtained from those organisms which naturally contain these genes, using methods known per se, for example via PCR on chromosomal DNA. Preference is given to enzymes. Host cells suitable for the particular protein must be determined experimentally in each individual case.

Suitable enzymes which can be produced with the aid of the method of the invention are primarily hydrolytic enzymes such as amylases, glucanases, proteases, lipases or cellulases, the enzymes naturally obtained from microorganisms such as bacteria or fungi being preferred. Similarly, it is also possible to obtain mixtures of such enzymes via coexpression in the same host cells. To this end, the corresponding genes may have been introduced into the host cells, for example, on different vectors or on the same vectors or may be encoded, at least partially, by the chromosome.

An example of a preferred enzyme is the enzyme α-amylase which can be produced according to the application examples of the present application. α-Amylase (E.C.3.2.1.1) is a hydrolase for α-1,4-glycosidic bonds as occur in amylose, amylopectin or glycogen; this reaction produces dextrins and β-1,6-branched oligosaccharides. These are among the most important industrially utilized enzymes of all. Their primary use is the production of glucose syrup. Other use examples are the uses as active components in detergents and cleaners, for treating raw materials in the manufacture of textiles, for the production of adhesives, for the production of sugar-containing food and/or food ingredients. An example of an amylase which is particularly intensively used industrially is the Bacillus licheniformis α-amylase which is sold by Novozymes A/S, Bagsvard, Denmark, under the trade name Termamyl®. The amylase obtained from B. subtilis and, respectively, B. amyloliquefaciens and disclosed in the US application U.S. Pat. No. 1,227,374 is sold by the same company under the name BAN®.

Another example of enzymes which can be produced according to the invention is β-glucanases. β-Glucanases are enzymes which hydrolytically cleave mixed glucans alternately linked by 1,3- and 1,4-β-glucosidic bonds to give oligosaccharides. They belong to the class of the endo-1,3-1,4-β-D-glucan 4-glucanohydrolases (EC 3.2.1.73; lichenases) or of the endo-1,3-β-D-glucosidases (EC 3.2.1.39; laminarinases). These mixed glucans are contained in virtually all cereal products. Enzymes which are capable of cleaving them are required especially in the food, beverage and animal feed industries, the textile industry and starch processing. In the beverage and brewing industry, for example, they serve to break down malt β-glucan and barley β-glucan, or they serve, when included in detergent or cleaner formulas, to break down corresponding soiling on textiles or solid surfaces. A Bacillus β-glucanase is disclosed, for example, in the application WO 99/06573 and its possible uses in detergents and cleaners are disclosed, for example, in the applications WO 99/06516 and WO 99/06515, respectively.

These two enzymes represent, by way of example, all other hydrolytic enzymes which include proteases, lipases and cellulases, but also nonhydrolytic enzymes, for example oxidases such as laccases, for example, since the type of production process is in principle unconnected to the type of reaction which is catalyzed by the particular enzymes.

In likewise preferred embodiments of this subject matter of the invention, all methods described thus far are used for the production of recombinant phytases, in particular of bacterial phytases, with in principle any, in each case expedient, combination of the mentioned method parameters.

Phytases (E.C. 3.1.3.26) hydrolyze phytates which are the salts, usually calcium or magnesium salts, of the phytic acids, i.e. of those organic compounds which serve as phosphate stores, in particular in plants. Phytases may be added, in particular in agricultural livestock management, to the feed of monogastric animals such as poultry or pigs and thus facilitate phosphate absorption in these animals. Thus fewer inorganic phosphates need to be added to the feed. These economically important enzymes, too, can be produced in a cost-effective manner via a method of the invention. A possible implementation of this embodiment is illustrated in example 4 of the present application.

Preference is given to appropriate methods for producing phytases, in which the Kil gene product is used to partially open the outer membrane. In addition, preference is given to placing this Kil protein under the control of a stationary-phase promoter, in particular one from the organism used for production, preferably the fic promoter from E. coli.

Owing to the molecular biological manageability, preferred embodiments for producing the bacterial phytases are characterized by the membrane-opening system, in particular the kil gene, being provided in a secretion cassette. For the abovementioned reasons, it is particularly advantageous to use a combined expression and secretion cassette. The further possible designs discussed above, for example regarding the location of these genetic elements, must be decided in each individual case on the basis of experimental data.

The E. coli phytase gene is produced naturally only under anaerobic conditions and with a low rate of expression. The use of the Bacillus amyloliquefaciens β-glucanase promoter enables a high rate of expression, moreover under aerobic conditions. This is substantiated by example 4 of the present application. Methods in which the bacterial phytases are expressed under the control of this promoter are preferred embodiments of this subject matter of the invention.

In example 4 of the present application, various Escherichia coli strains have been tested. All of them characterize embodiments of the present invention. A particularly high rate of product formation was achieved using the strain E. coli BL21 (DE3). This strain characterizes particularly preferred embodiments for the inventive production of bacterial phytases.

Example 4 and FIG. 4 of the present application also describe the manner in which various vectors having a combined expression and secretion cassette can be constructed. The vector pPhyt109 contains the kil gene under the control of the fic promoter (compare Miksch, G. et al., (1997), Arch. Microbiol., Vol. 167, pp. 143-150); and the vector pPhyt119/4 contains the kil gene under the control of the bgl A promoter; the latter is additionally distinguished from the former by the absence of an interposon upstream from the kil gene. Both vectors characterize preferred embodiments of this subject matter of the invention.

Owing to their universal transferability to various host organisms, the secretion cassettes already described further above which contain the genetic elements responsible for the membrane-opening properties of the membrane-opening system represent separate subject matters of the invention. This is because their importing into a bacterial strain which already expresses a transgene and encloses this transgene, for example, in inclusion bodies or secretes it into the periplasm converts said bacterial strain to a secretion-competent bacterial strain. And, owing to the teaching provided by this application, it is to be expected that, just by transferring a secretion competence of the invention into an established, transgene-expressing Gram-negative bacterial strain, higher rates of product formation and easier obtainability of the product from the medium in which the producing material strains are cultured will be achieved without further modifications.

Owing to the examples carried out, secretion cassettes containing the colicin system from E. coli, in particular the gene for the Kil protein, and/or a system under the control of the fic promoter are preferred embodiments of this subject matter of the invention. Alternative embodiments which are likewise included in this subject matter of the invention have already been discussed further above.

Further preference is given to those secretion cassettes which additionally contain, immediately upstream or downstream, an expression cassette which consists of the transgene and a promoter as the control element thereof. This preferably includes in particular promoters which are not necessarily to be induced from the outside, preferably constitutive promoters, and particularly preferably the Bacillus amyloliquefaciens β-glucanase promoter (bgl promoter).

These expression cassettes make possible the production of any protein. Owing to their economic importance, preference is given to those for enzymes, particularly for hydrolases, and among these in particular those for amylases, glucanases, proteases, lipases, cellulases or for bacterial phytases.

Vectors containing an above-described secretion cassette, which replicate in Gram-negative bacteria, i.e. which can be recognized by the particular cellular systems, represent a separate subject matter of the invention, since they are used to realize the present invention. This applies increasingly to the increasingly preferred forms of the above-described secretion cassettes, in particular to those vectors which additionally contain an expression cassette. That is because these two elements impart to the host cells all features essential to the invention, namely synthesis of the protein of interest and its export into the surrounding medium via a system which partially opens the outer membrane of the Gram-negative bacteria.

This includes, owing to the scientific importance of Escherichia coli and Klebsiella, preferably those expression vectors which are suitable for use in said species. For this purpose, they must be equipped with the appropriate genetic elements such as, for example, the particular origin of replication and, suitably, with selection markers.

The vectors pAmy63, pPhyt 109 and pPhyt119/4 in particular represent embodiments of this subject matter of the invention. They are preferably employed for production of α-amylase, β-glucanase and phytase. On the basis of the examples included in the present application and starting from the same or other, for example commercially available, vectors, it is possible to construct corresponding expression vectors which realize the subject matter of the invention in the same manner. All vectors which can be derived from these expression vectors and thus share with these vectors the essential genetic elements are likewise within the scope of protection. This applies in particular to those which can be derived from any of these vectors by replacement of the gene to be expressed, since, as already explained above, it is not essential to the invention which proteins are actually involved, since they are exported unspecifically via the membrane-opening system.

The examples illustrate how such vectors can be prepared. Variations of these vectors, for example integration of a different membrane-opening system, of different promoters or different transgenes, are possible according to the methods familiar to the skilled worker, these being indicated, for example, also in the manual by Fritsch, Sambrook and Maniatis, “Molecular cloning: a laboratory manual”, Cold Spring Harbour Laboratory Press, New York, 1989.

However, cloning vectors containing any of the above-described expression cassettes are also embodiments of this subject matter of the invention. They represent in a way the possible genetic implementations of the present invention. They serve, for example, to store but also to copy the above-described genetic elements, for example in vivo via transformation into different bacterial strains or in vitro as template for PCR. They serve, in particular, to modify the relevant elements, in particular to optimize them for the specific case. An optimization of this kind may be, for example, a promoter analysis, i.e. determination of a promoter individually suitable for the transgene. Thus these elements may be, for example, point-mutated via PCR (polymerase chain reaction) or combined with other elements. Another possible modification is to introduce a region on a vector, which is flanked, for example, by transposon elements, into a host cell and to enable in vivo excision and integration into the host chromosome. In this way, new secretion-competent bacterial strains in which the expression cassette and/or secretion cassette are located chromosomally are obtained. In analogy thereto, importing via homologous recombination is also possible.

A secretion cassette flanked by the insertion sequences of a transposon is described in Appl. Microbiol. Biotechnol. (1997), Vol. 47, pp. 530-536) and used in the examples of the present application. Example 1 illustrates the construction of secretion strains in which secretion competence is integrated into the bacterial chromosome via homologous recombination.

The secretion cassette can also be transferred to bacterial species other than those in which cloning of the gene to be expressed has taken place by making use of conjugation processes as can be observed naturally also between Gram-negative bacteria of different species, for example between E. coli and Klebsiella.

Bacterial strains which are used to realize the present invention form a separate subject matter of the invention. They include, for example, those Gram-negative bacteria which carry any of the above-described secretion cassettes located on a vector, since their culturing makes possible both synthesis and secretion and thus the inventive production of the proteins of interest. Location on a vector makes possible a flexible molecular biological development of said strains and broad regulation of the copy numbers of the active genetic elements. Owing to the knowledge illustrated above and to the successful experiments documented in the examples of the present application, preferred strains are coliform bacteria, very particularly those of the genera Escherichia coli and Klebsiella and among these in particular derivatives of E. coli K12 or E. coli B or of Klebsiella platicola.

Among these, preference is in turn given to those strains which can be derived from E. coli BL21 (DE3), E. coli RV308, E. coli DH5α, E. coli JM109, E. coli XL-1 or from Klebsiella platicola (Rf), in particular from the strain deposited with application number DSM 14225, for example by transformation using an appropriate secretion and/or expression cassette.

In principle, this can already be applied to bacterial strains which naturally produce particular proteins whose production is of economic interest, but applies in particular to bacterial strains which are characterized in that they additionally contain an expression vector with a promoter and a gene regulated by said promoter.

Among these bacterial strains, preference is given to those which have been obtained after transformation with any of the vectors illustrated above, in particular pPhyt 109 or pPhyt119/4, or any vector which can be derived from these vectors. This applies in particular to those which make possible the production of other economically interesting proteins.

Another embodiment of this subject matter of the invention is represented by Gram-negative bacterial strains in which one of the above-described secretion cassettes is located chromosomally, since this chromosomal location makes it possible for these genetic elements to be established in a more stable way over several generations. Said bacterial strains include, for the reasons stated above, coliform bacteria, and among these those which can be derived from representatives of the genera Escherichia coli and Klebsiella, preferably from derivatives of E. coli K12 or E. coli B or Klebsiella planticola, very particularly from those of the strains E. coli BL21 (DE3), E. coli RV308, E. coli DH5α, E. coli JM109, E. coli XL-1 or of K. planticola (Rf).

Among these strains, preference is given in each case to those which express the recombinant protein under the control of a promoter which is not necessarily to be induced from the outside, preferably a constitutive promoter, and particularly preferably the Bacillus amyloliquefaciens β-glucanase promoter (bgl promoter). As explained above, this is because a high basal rate of transcription and translation, presumably in such a way that the protein formed is continuously removed from the reaction equilibrium via the partially opened membrane, in the end makes possible a high rate of production, i.e. a high concentration of the protein of interest in the surrounding medium.

A very particularly preferred embodiment of this subject matter of the invention is represented by derivatives of the microorganism deposited with the application number DSM 14225.

Preference is also given to those microorganisms which are characterized in that they have been obtained after transformation with any of the above-described vectors. These may be, for example, cloning vectors which have been introduced into a random bacterial strain for storage and/or modification. These steps are common in the storage and development of relevant genetic elements. Since it is possible to transfer the genetic elements in question from these microorganisms immediately into Gram-negative bacteria suitable for expression, the transformation products above are also realizations of the relevant subject matter of the invention.

Fermentation methods are well known per se in the prior art and represent the actual industrial production step; followed by a suitable purification method. Compared with the actual protein production, they represent a technical development and, when having features of the invention, form a separate subject matter of the invention. Thus all methods of fermentation of Gram-negative bacteria are claimed which produce a recombinant protein which is at least partially secreted into the medium surrounding said bacteria with the aid of a system which partially opens the outer membrane of said bacteria, which methods are characterized in that the recombinant protein is expressed under the control of a promoter from a Gram-positive organism, preferably from an organism of the genus Bacillus, which promoter does not naturally regulate the corresponding gene or a gene highly homologous to this gene.

All fermentation methods which are based on any of the above-described methods for producing the recombinant proteins are correspondingly preferred embodiments of this subject matter of the invention.

In this connection, the in each case optimal conditions for the production methods used, for the host cells and/or for the proteins to be produced must be determined experimentally on the basis of the previously optimized culture conditions of the relevant strains according to the knowledge of the skilled worker, for example with respect to fermentation volume, media composition, oxygen supply or stirrer speed. Example 4 of the present application provides an indication of this. Here too, the fermentation conditions chosen have been influenced by knowledge previously obtained on the basis of the shaker culture.

Of the fermentation methods, preference is given to those which are characterized in that the protein of interest is expressed under the control of a promoter which is not necessarily to be induced from the outside, preferably of a constitutive promoter and in particular of the Bacillus amyloliquefaciens β-glucanase promoter, and/or is released under the influence of the Kil protein, since this combination ensures, as the examples of the present application prove, a particularly high concentration of the protein in question in the culture medium.

Preference is increasingly given to those methods which are characterized in that the above-described preferred bacteria are used.

Preference is given to those fermentation methods which are characterized in that the fermentation is carried out via a continuous supply strategy. In this case, as demonstrated, for example, on the basis of the fermentation of example 4, the media components which are consumed by the continuous cultivation are continuously fed; this is also known as a continuous feed strategy. This makes it possible to achieve considerable increases both in cell density and in dry biomass and/or especially in the activity of the protein of interest.

Similarly, the fermentation may also be designed so as to filter out undesired metabolic products or to neutralize them by adding buffer or the appropriate counterions.

The protein produced can be harvested from the fermentation medium subsequently. This fermentation method is preferred compared with product preparation from the dry mass.

In contrast, however, preference is given to those methods which are characterized in that the protein produced is continuously removed during fermentation. This makes it possible, in particular in combination with the above-discussed continuous feed strategy and/or the possibility of continuously removing metabolic products from the medium, to run a fermentation over a long period. The latter is supported by the fact that the host cells need not be disrupted, i.e. destroyed, in order to obtain protein. A culture of this kind may be carried out, for example, via immobilized producers and represents a usually more cost-effective alternative compared to a batch culture.

### EXAMPLES

All molecular biological steps follow standard methods as indicated, for example, in the manual by Fritsch, Sambrook and Maniatis, “Molecular cloning: a laboratory manual”, Cold Spring Harbour Laboratory Press, New York, 1989.

### Example 1

Production of α-amylase by E. coli BL21 (DE3)

Preliminary Experiments

In order to culture the strain E. coli BL21 (DE3) (Stratagene, La Jolla, USA), the following media, prepared according to Fritsch, Sambrook and Maniatis (see above), were compared with one another in preliminary experiments:

complete medium LB+2% NaCl+0.2% glycerol,

complete medium TB+2% NaCl and

minimal medium M9+2% NaCl+0.2% glycerol.

All cultures were kept in a volume of 30 ml in 300 ml Erlenmeyer flasks with baffles and incubated with shaking at 175 rpm; the temperature was 37° C. in each case. The highest biomass yields were obtained in TB medium+2% NaCl, which medium was therefore used for the subsequent experiments under the same culture conditions.

Construction of an Expression Vector with Secretion Cassette

Construction of the Tn5-derived secretion cassette per se is described in Appl. Microbiol. Biotechnol., Volume 47 (1997); pp. 143-150. It contains the following elements: IS50_{R}, kanamycin-resistance gene (Km), kil gene (kil), fic promoter (Pₖᵢₗ), multiple cloning site, an omega interposon (Ω-Cm; according to Prentki, P., Frisch, H. M. (1984), Gene, Vol. 29, pp. 303-313) as terminator, mobility gene (mob) and IS50_{L}. Thus it makes possible, via the fic promoter, a stationary phase-dependent activation of the kil gene product and, depending thereon, partial lysis of the cells. It provides a multiple cloning site for integration of the gene of interest and of a promoter responsible for this gene. The ends originating from Tn5 (IS_{R}, IS_{L}: insertion sites or inverted repeats) allow integration both into plasmids and into the bacterial chromosome. This actual secretion cassette is to be distinguished from the element later referred to as “complete” secretion cassette which additionally contains the transgene and the promoter regulating this transgene.

The vector pUC19 (Pharmacia, Freiburg) combined the Bacillus amyloliquefaciens α-amylase gene with the bgl promoter. This promoter is constitutive and need not be activated by induction. It was isolated from the plasmid pLF3 (in Appl. Microbiol. Biotechnol., Volume 47 (1997), pp. 120-126) by PCR using the primers 5′AAC GAA TTC AAC GAA GAA TCGCTG CAC3′ (with EcoRI restriction cleavage site) and 5′TCG CGG ATC CTT ACC CCT TTT TTG AAC ACG C3′ (with the BamHI restriction cleavage site) and integrated into the EcoRI/BamHI site of the pUC19 vector.

Subsequently, the α-amylase gene was obtained by means of PCR from chromosomal DNA of Bacillus amyloliquefaciens DSM7 (corresponds to ATCC 23350; sequence according to EMBL sequence database (Cambridge, United Kingdom) under accession number J01542). It was carried out using the primers PA02 (5′TTT GGA TCC GAA AAT GAG AGG3′) and PA03 (5′ATT GGG AGC TCC TAC GAT CGC3′) amplified. The gene obtained was cloned into the vector PGEM Teasy (Promega, Madison, Wis., USA). With correct orientation of the insert, it was possible to obtain from this vector the α-amylase gene on a BamhI/SalI fragment and to clone it into the abovementioned pUC19 downstream of the bgl promoter.

As a result, the vector pAmy58 which enables expression but not secretion was obtained. The secretion cassette was inserted as above as PvuII fragment into the SspI restriction cleavage site upstream of the β-lactamase gene of the pUC19 vector. As a result, the vector pAmy63 with complete secretion cassette was obtained. The corresponding promoter structure is depicted in FIG. 1.

This vector was used to transform preparations of E. coli BL21 (DE3) according to standard methods and the strain E. coli BL21 (DE3) (pAmy63) was obtained. This strain was cultured in the same way as the starting strain E. coli BL21 (DE3).

Preliminary Assay for Amylase Production

A qualitative assay for an α-amylase produced by this strain is the plate assay in which 5 μl of the supernatant of the liquid culture are applied to LB agar plates containing 1% starch (Sigma, Deisenhofen, Germany). As a result, haloues with sharp outlines are obtained after just a few hours of incubation, and a quantitative distinction is already possible via the halo diameter and the sharpness of the halo outlines. Even single colonies of amylase-positive clones form readily visible halos on starch-containing agar plates.

Determination of α-amylase Activity

Quantitative determination of α-amylase was carried out by measuring amylase activity by means of SIGMA-amylase test reagent from SIGMA DIAGNOSTICS (St. Louis, USA; product No. 577). According to the directions, 20 μl of sample were used. The measurements were carried out on a 30 ml culture in 300 ml Erlenmeyer flasks with baffles, which were kept on a rotary shaker at 175 rpm and 37° C. The results are obtained in IU/ml as defined according to Fresenius Z. Anal. Chem., Vol. 301 (1980),

TABLE 1
Ability of the strains constructed to produce
α-amylase
Optical density and α-amylase activity (in
IU/ml) in the periplasm (PP) and in the culture medium
(S), using the bgl promoter, in each case at two
different times (13 h and 18 h after inoculation)

| | | | | Strain with vectorial | | |
|---|---|---|---|---|---|---|
| | Control strain: | | | secretion cassette: | | |
| | E. coil BL21 (DE3) | | | E. coli BL21 (DE3) | | |
| Time | pAmy58 | | | pAmy63 | | |
| [h] | OD₆₀₀ | PP | S | OD₆₀₀ | PP | S |
| 13 | 14.3 | 0.10 | 0.01 | 14.5 | 0.15 | 1.16 |
| | | | | | | (88%) |
| 18 | 15.7 | 0.14 | 0.01 | 12.4 | 0.11 | 1.22 |
| | | | | | | (92%) |

The control strain shows a base rate of periplasmic but not secreted enzyme activity. In the case of vectorially encoded expression, the enzyme activity is increased 1.5-fold and 12-fold in the periplasm and, respectively, in periplasm and supernatant combined. After another 5 h, in each case a further part of the periplasmic activity has been released into the surrounding medium so that, however, the vectorial location leads to only an 8.9-fold increase in enzyme activity.

Thus, a surprisingly positive effect occurs when using the bgl promoter. The vectorial location of the secretion cassette leads to a secretion of the protein into the surrounding medium. The extracellular portion is 88% at 13 h after inoculation and even 92% after 18 h, i.e. the proportion of α-amylase in the periplasm decreases and the proportion in the culture medium increases with longer culturing time.

### Example 2

Production of α-amylase by E. coli RV308

The strain E. coli RV308 (ATCC 31608) was tested in the Hans-Knöll-Institut für Naturstoff-Forschung in Jena, Germany, and is described in J. Mol. Biol., Vol. 139 (1980), pp. 147-161. It is distinguished by not producing acetate (Appl. Microbiol. Diotechnol., Vol. 46 (1996), pp. 524-532). The same culture conditions and detection reactions as in application example 1 are suitable here.

According to the procedure described in application example 1, the E. coli strain RV308 was transformed with the vector pAmy63 (with bgl promoter) which enables expression and secretion of α-amylase. As a result, this strain E. coli RV308 pAmy63 containing a vectorially encoded complete secretion cassette, i.e. a secretion cassette also containing the transgene and the regulating promoter, was obtained. The results obtained therewith are listed in table 2.

TABLE 2
α-Amylase production by E. coli RV308 pAmy63.
α-Amylase activity (in IU/ml) was measured in
the periplasm (PP) and in the supernatant
(S)/13 and 18 h after inoculation.

| Time | | | |
|---|---|---|---|
| [h] | OD₆₀₀ | PP | S |
| 13 | 12.4 | 0.06 | 0.15 |
| | | | (68%) |
| 18 | 10.4 | 0.01 | 0.15 |
| | | | (94%) |

With bgl promoter-dependent expression and secretion according to the invention, this E. coli strain likewise shows detectable α-amylase production and secretion and is thus an alternative to E. coli BL21 (DE3).

### Example 3

Production of α-amylase by Klebsiella planticola (Rf)

Klebsiella planticola (Rf) is a rifamycin-resistant strain derived from Klebsiella planticola by spontaneous mutation (Appl. Microbiol. Biotechnol., Vol. 51 (1999), pp. 627-632). The same culture conditions and detection reactions as in examples 1 and 2 are suitable for this example. Similarly to example 1, the actual expression cassette without transgene and promoter was integrated into the bacterial chromosome for this application example and the expression cassette was made available on a separate vector.

Preparation of the secretion-competent Klebsiella strain is described in Appl. Microbiol. Biotechnol. (1999), Vol. 51, pp. 627-632 and is denoted Klebsiella planticola (Rf)-FIC3/19. For this purpose, a transposon with secretion cassette and with the genes required for plasmid mobilization had been constructed, starting from the Tn5 derivative Tn5-B13, so that, in the presence of this transposon, those plasmids which do not carry their own mobility genes can also be mobilized. Said transposon which is located on the vector pBR325 and is referred to as Tn5-KIL3 had been transferred from the mobilizing E. coli strain S17.1 into K. planticola. Since pBR325 does not replicate in Klebsiella, all Km-resistant transconjugants represent transposition events. It had been shown there that Tn5-KIL3 integrates randomly at different sites, but in each case only as a single copy, into the bacterial chromosome. In the study mentioned, the plasmid pRS201L-Tc with the gene for β-glucanase had been transferred by conjugation into those transconjugants. The latter are distinguished by carrying a secretion cassette comprising mobilization genes, the kil gene and a kanamycin-resistance gene integrated into the chromosome.

For the present invention, the plasmid pRS-Amy has been transferred by conjugation into the secretion strain Klebsiella planticola (Rf)-FIC3/19. As described in FIG. 2, this vector is derived from plasmid pRS201. This vector which is in turn derived from RSF1010 and which has a wide host range is required because E. coli vectors cannot replicate in Klebsiella without the appropriate origin of replication (ori). The vector pRS201 was reduced in size by deleting unnecessary parts in the form of an approx. 2 kb fragment and then an interposon containing a tetracycline-resistance gene was integrated into the EcoRI cleavage site. After deleting the approx. 1.4 kb fragment carrying the kanamycin-resistance gene, the PvuII/PstII fragment from pAmy58 (see example 1) was incorporated. This fragment contains the bgl promoter and the α-amylase gene under the control of said promoter.

This vector pRS-Amy was firstly transformed into E. coli S17-7 and mobilized from there into K. planticola via conjugation so that again a Gram-negative organism contained at the same time a chromosomally encoded colicin system and a bgl promoter-controlled gene located on a vector. The strain obtained was denoted Klebsiella planticola (Rf)-FIC3/19. The entire procedure is summarized in FIG. 3. The control used was a transformant which had been obtained by transferring the same plasmid into the starting strain Klebsiella planticola, i.e. without secretion competence.

Replica-plating on starch-containing LB agar plates shows secretion-competent transconjugants. α-Amylase activity was detected and quantified as described in application example 1. The activity data obtained by quantitative measurements are summarized in table 3.

TABLE 3
α-Amylase production by Kiebsiella planticola
Optical density and amylase activity (IU/ml)
in the periplasm (PP) and the supernatant (S)
in K. planticola strains with and without
secretion.

| | Kiebsiella | | | Kiebsiella | | |
|---|---|---|---|---|---|---|
| | planticola (Rf) | | | planticola (Rf) - | | |
| Culturing time | pRS-Amy (control) | | | FIC3/19pRS-Amy | | |
| [h] | OD₆₀₀ | PP | S | OD₆₀₀ | PP | S |
| 13 | 12.4 | 0.22 | 0.01 | 20.4 | 0.15 | 0.06 |
| 18 | 12.8 | 0.17 | 0.01 | 18.2 | 0.11 | 0.08 |

According to this, the periplasmically detectable enzyme activities are not increased to a detectable extent compared to the controls without secretion cassette, but the activities secreted into the supernatant are. This demonstrates that the principle of the invention also applies to the Gram-negative organism Klebsiella planticola.

The secretion-competent bacterial strain Klebsiella planticola (Rf)-FIC/19, prepared according to this example and according to Appl. Microbiol. Biotechnol. (1999), Vol. 51, pp. 627-632, was deposited with the Deutsche Sammlung von Mikroorganismen und Zellkulturen GmbH, Brunswick, Germany with the number DSM 14225 on Apr. 09, 2001.

### Example 4

Expression of Phytase by E. coli

Fusion of the Phytase Gene with an Effective Promoter and Construction of an Expression Vector

The gene for E. coli phytase including the ribosomal binding site was amplified from the plasmid pPH251 (Greiner, R. et al. (1993), Arch. Biochem. Biophys., Vol. 303, pp. 107-113) by means of PCR, and the amplified region had been provided with the restriction cleavage sites BamHI and PstI. The phytase gene was then fused with the promoter of Bacillus amyloliquefaciens β-glucanase (P_{bglA}), and both were integrated together into the high copy number vector pUC19. In addition, the secretion function was integrated into the plasmid by cloning of a cassette. The cassette was applied in the form of two different structures (FIG. 4):

1. Cassette with kil gene under the control of the fic promoter (compare Miksch, G. et al. (1997), Arch. Microbiol., Vol. 167, pp. 143-150); this type of cassette is contained in the vector pPhyt109.

2. Cassette with kil gene under the control of the bglA promoter; this type of cassette differs from the one under 1. in addition by the absence of an interposon upstream of the kil gene; this type of cassette is contained in the vector pPhyt119/4.

Promoter-Dependent Phytase Expression

The phytase was overexpressed only under secretion conditions, i.e. in the presence of the secretion cassette described.

TABLE 4
Phytase activity (in % of maximum) in E. coli
BL21 (DE3).
Culture conditions: 30 ml shaker culture in
300 ml Erlenmeyer flasks with baffles; TB medium;
temperature: 37° C.; rotary shaker at 150 rpm.

| | P_{bglA} |
|---|---|
| Medium | 75.4 |
| Periplasm | 25.4 |

Secretion-Dependent Phytase Expression

FIG. 5 shows the kinetics of total, extracellular, periplasmic and cytoplasmic phytase activities depending on secretion during a batch fermentation. The two strains used here differ in that the secretion variant (bottom) contained the secretion cassette on the expression vector, while the expression vector of the control strain (top) lacked said secretion cassette. FIG. 5 shows that total phytase activity and phytase activity in the medium rapidly increased from the late exponential phase onward, while in the control no activities or extremely small activities were observed during the entire culturing time.

E. coli Strain-Dependent Phytase Expression

In order to study the influence of the host strain genome on phytase activity, the plasmid pPhyt19/4 was transformed into the following E. coli strains: BL21 (DE3), JM109 and TG1 (Stratagene, La Jolla, USA).

Phytase activities in the culture medium were compared after 24 or 48 h of culturing. Table 5 shows that the strain BL21 (DE3) makes possible a markedly higher extracellular phytase production compared with the other strains.

TABLE 5
Phytase activity (in % of maximum) in the
supernatant of E. coli strains containing the
plasmid pPhyt119/4 as a function of the
culture time.
Culture conditions: 30 ml shaker culture in 300 ml
Erlenmeyer flasks with baffles; TB medium (complete
medium); temperature: 37° C; rotary shaker at 150 rpm.

| | BL21 (DE3) | JM109 | TG1 |
|---|---|---|---|
| 24 h culture | 88 | 27 | 25 |
| 48 h culture | 100 | 36 | 56 |

Influence of the Fermentation Method on Phytase Expression and Secretion

The strain BL21 (DE3) pPhyt109 was assayed in a 7 l fermenter with respect to cell density and phytase activity. The following two methods were compared with one another: batch culture and continuous supply method. In the continuous supply method, a synthetic medium suitable for high cell density fermentations (Horn, U. et al., (1996), Appl. Microbiol.,Vol. 46, pp. 524-532) was used and addition of glucose and ammonium sulfate was controlled via oxygen saturation (PO₂). The continuous feed started at 60% oxygen saturation and was interrupted at 30%.

FIG. 6 shows that, as measured by optical density and dry biomass, the continuous feed strategy achieves substantially higher cell densities and more than three times higher phytase yields (total phytase activity and phytase activity in the medium) than the batch culture.

### DESCRIPTION OF THE FIGURES

FIG. 1: Genetic structure of the bgl promoter for controlling the α-amylase gene.

FIG. 2: Construction of the vector pRS-Amy from the vector pRS201.

FIG. 3: Construction of secretion strains in K. planticola (identical to FIG. 1 in Appl. Microbiol. Biotechnol. (1999), Vol. 51, pp. 627-632)

FIG. 4: Genetic structure of the vectors pPhyt109 (top) and pPhyt119/4 (bottom) used for extracellular production of E. coli phytase.

FIG. 5: Phytase production and phytase secretion into the culture medium during culturing, according to example 4; determined for a batch fermentation in a 7 l fermenter.

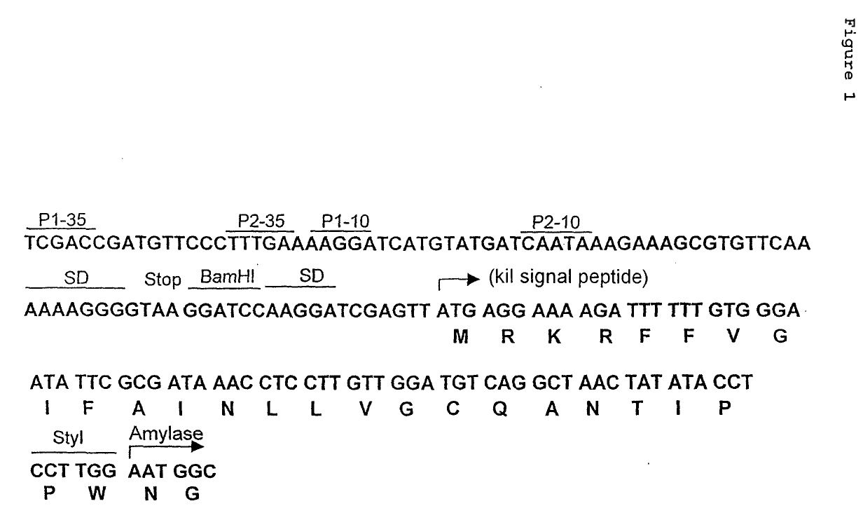

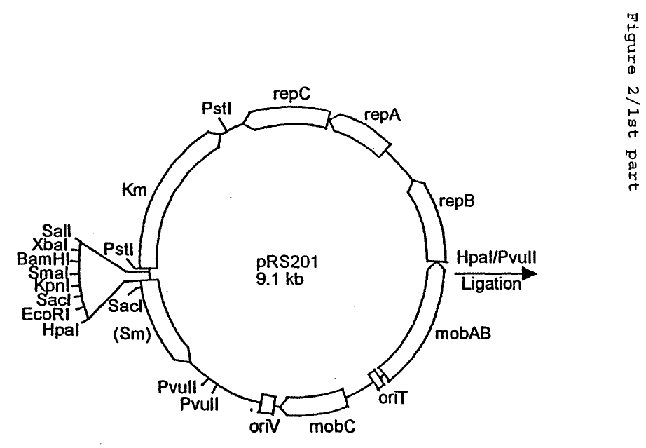

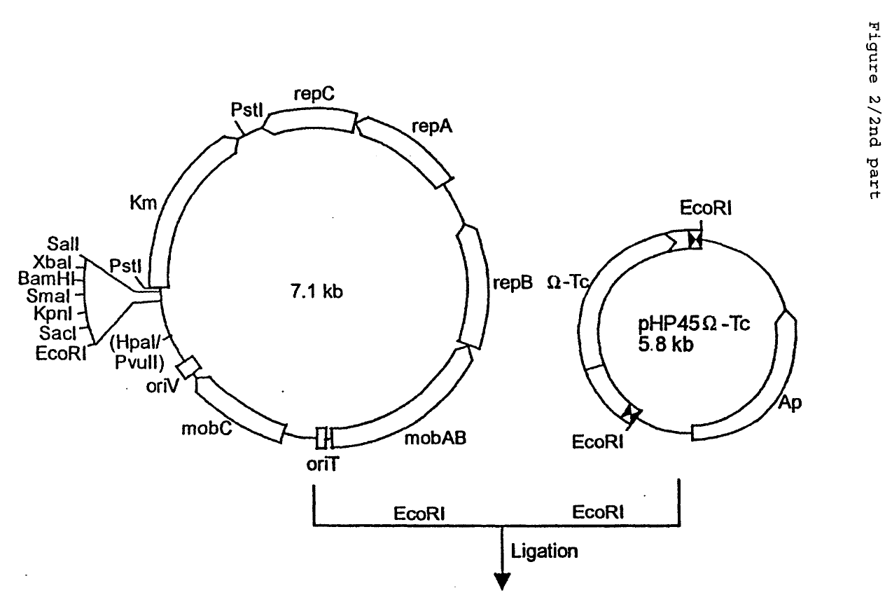

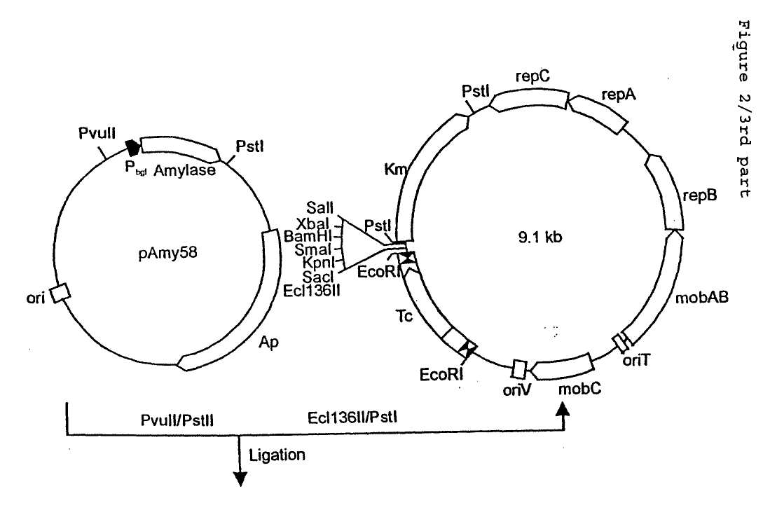

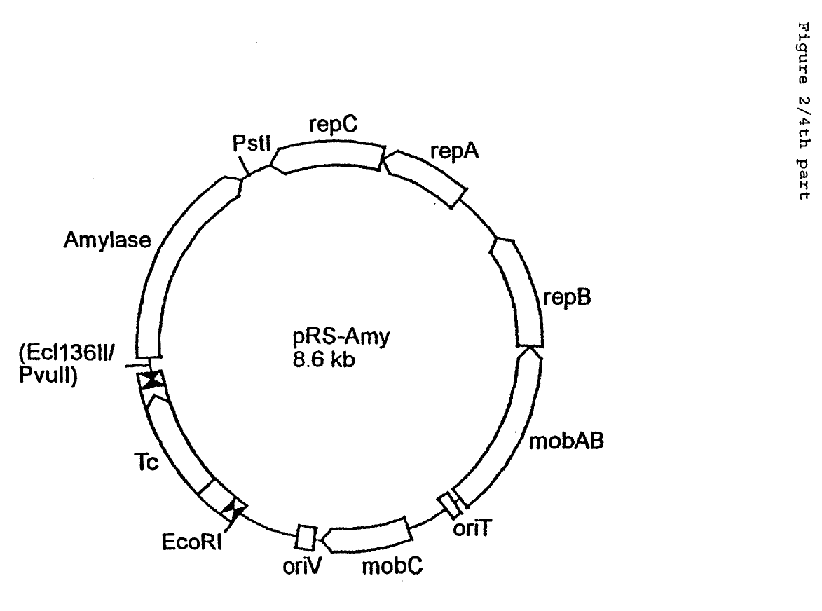

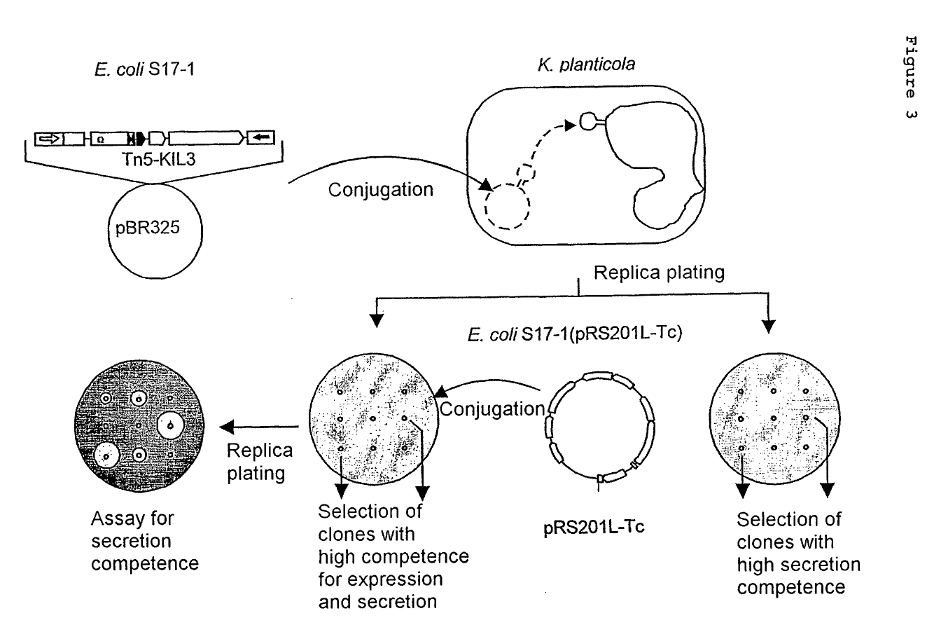

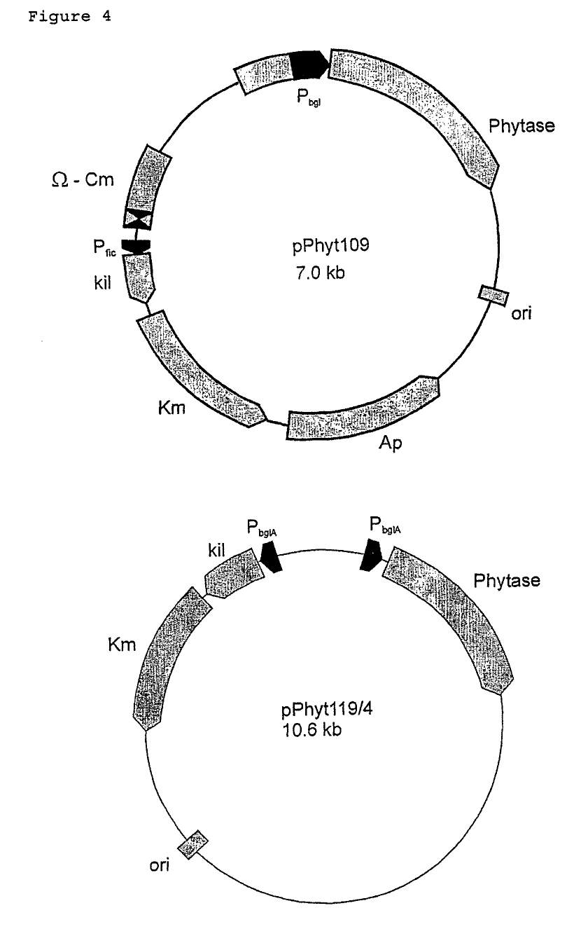

Optical density of cell suspension (cell density): empty circles; y axis, left scale;

Phytase activity: in each case in U/ml; y axis, right scale;

Total phytase activity: filled squares;

Phytase activity in medium: filled circles;

Time: in h; x axis.

Medium: synthetic medium according to Horn, U. et al., (1996), Appl. Microbiol., Vol. 46, pp. 524-532; temperature: 37° C.

top: strain BL21 (DE3) pPhyt106 (without secretion cassette);

bottom: strain BL21 (DE3) pPhyt109 (with secretion cassette).

In addition, the dry biomass (in mg/ml; empty triangles; y axis, left scale), periplasmic phytase activity (filled triangles pointing upward) and cytoplasmic phytase activity (filled triangles pointing downward) are indicated here.

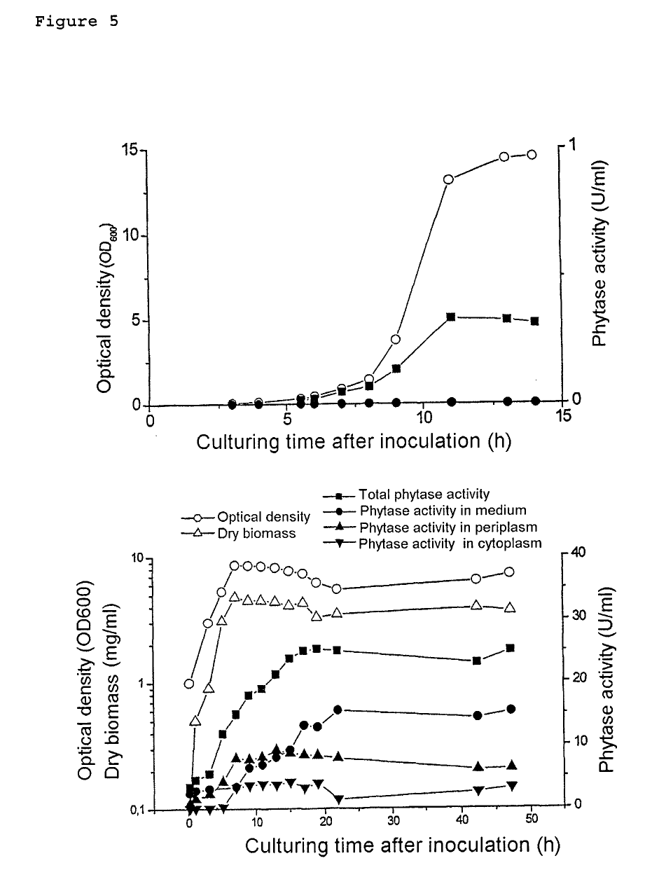

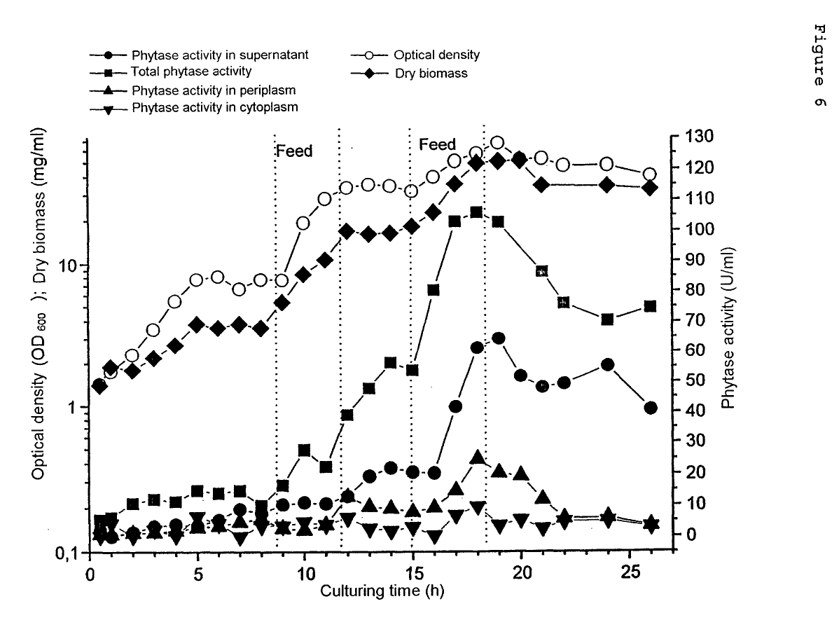

FIG. 6: Fermentation of the strain BL21 (DE3) pPhyt109 in a 7 l fermenter in a continuous supply process; the continuous supply phase is indicated by an arrow. Culturing conditions and representation as in FIG. 5.

1. A method for producing a recombinant protein by Gram-negative bacteria, which protein is at least partially secreted into the medium surrounding said bacteria with the aid of a system which partially opens the outer membrane of these bacteria, characterized in that the recombinant protein to be produced is expressed under the control of a promoter from a Gram-positive organism, preferably from an organism of the genus Bacillus, which promoter does not naturally regulate the corresponding gene or a gene highly homologous to this gene.

2. The method as claimed in claim 1, characterized in that the Gram-negative bacteria are coliform bacteria, in particular those of the genera Escherichia coli or Klebsiella.

3. The method as claimed in claim 2, characterized in that the coliform bacteria are derivatives of Escherichia coli K12, of Escherichia coli B or Klebsiella planticola, very particularly those of the strains Escherichia coli BL21 (DE3), E. coli RV308, E. coli DH5α, E. coli JM109, E. coli XL-1 or Klebsiella planticola (Rf).

4. The method as claimed in claim 3, characterized in that the microorganism is the strain deposited with the application number DSM 14225 or a derivative of this strain.

5. The method as claimed in any of claims 1 to 4, characterized in that the system which partially opens the outer membrane is the E. coli colicin system, in particular the Kil protein and/or a system under the control of the fic promoter or of another stationary-phase promoter.

6. The method as claimed in any of claims 1 to 5, characterized in that the expression promoter is a promoter which need not necessarily be induced from the outside, preferably a constitutive promoter and particularly preferably the Bacillus amyloliquefaciens β-glucanase promoter.

7. The method as claimed in any of claims 1 to 6, characterized in that secretion competence is mediated via a secretion cassette, in particular one which has been integrated into the chromosome.

8. The method as claimed in any of claims 1 to 7, characterized in that the expression cassette and the secretion cassette are located on different replicons.

9. The method as claimed in any of claims 1 to 7, characterized in that the expression cassette is located on the same replicon as the secretion cassette, in particular in the form of the expression cassette being located immediately upstream or downstream of the secretion cassette.

10. The method as claimed in any of claims 1 to 9, characterized in that the expression cassette and/or the secretion cassette are located on a plasmid which can replicate autonomously, preferably on the same plasmid.

11. The method as claimed in any of claims 1 to 10, characterized in that the protein is an enzyme.

12. The method as claimed in claim 11, characterized in that it is a hydrolase, in particular an amylase, glucanase, protease, lipase or cellulase.

13. The method as claimed in any of claims 1 to 12, characterized in that recombinant proteins phytases, in particular bacterial phytases, are produced.

14. The method as claimed in claim 13, characterized in that the phytase is secreted by using the E. coli kil gene under the control of an E. coli stationary-phase promoter, preferably the fic promoter.

15. The method as claimed in claim 13 or 14, characterized in that the membrane-opening system, in particular the kil gene, is provided via a secretion cassette.

16. The method as claimed in any of claims 13 to 15, characterized in that the gene of the phytase is under the control of the Bacillus amyloliquefaciens β-glucanase promoter.

17. The method as claimed in any of claims 13 to 16, characterized in that the host strain used is Escherichia coli BL21 (DE3).

18. The method as claimed in any of claims 13 to 17, characterized in that the expression vectors used are the vectors pPhyt109 or pPhyt119/4 or vectors derived therefrom.

19. A secretion cassette which possesses the genetic elements responsible for the membrane-opening properties of the membrane-opening system, in particular the E. coli colicin system and/or a stationary-phase promoter, very particularly the gene for the Kil protein and/or an E. coli stationary-phase promoter including, in particular, the fic promoter.

20. The secretion cassette as claimed in claim 19, which additionally contains immediately upstream or downstream an expression cassette containing the transgene and a promoter as the control element of said transgene, including, in particular, a promoter which need not necessarily be induced from the outside, preferably a constitutive promoter and particularly preferably the Bacillus amyloliquefaciens β-glucanase promoter.

21. The secretion cassette as claimed in claim 20, which contains as transgene the gene for an enzyme, preferably that of a hydrolase, in particular that of an amylase, glucanase, protease, lipase or cellulase or that of a bacterial phytase.

22. A vector which can replicate in Gram-negative bacteria and which contains a secretion cassette as claimed in any of claims 19 to 21, in particular a vector which additionally contains the expression cassette.

23. The expression vector as claimed in claim 22 for Gram-negative bacteria, in particular for coliform bacteria, among these in particular for those of the species Escherichia coli or Klebsiella, very particularly any of the vectors pAmy63, pPhyt 109 or pPhyt119/4 or a vector which can be derived from any of these vectors, in particular by replacing the gene to be expressed.

24. A cloning vector containing a secretion cassette as claimed in any of claims 19 to 21.

25. A Gram-negative bacterial strain which carries a secretion cassette as claimed in any of claims 19 to 21 in a vectorial location, in particular a coliform bacterial strain, very particularly of the genera Escherichia coli and Klebsiella, and among these in particular derivatives of E. coli K12, E. coli B or Klebsiella platicola.

26. The bacterial strain as claimed in claim 25, characterized in that it is derived from E. coli BL21 (DE3), E. coli RV308, E. coli DH5α, E. coli JM109, E. coli XL-1, from Klebsiella platicola (Rf) or from the strain deposited with the application number DSM 14225.

27. The bacterial strain as claimed in claim 25 or 26, characterized in that it additionally contains an expression vector with a promoter and a gene regulated by said promoter.

28. The bacterial strain as claimed in any of claims 25 to 27, characterized in that it has been obtained after transformation with any of the vectors as claimed in any of claims 22 to 24.

29. A Gram-negative bacterial strain which carries a secretion cassette as claimed in any of claims 19 to 21 in a chromosomal location, in particular coliform bacteria, and among these in particular strains of Escherichia coli or Klebsiella, preferably of derivatives of Escherichia coli K12 or Escherichia coli B or Klebsiella planticola, very particularly of those of the strains Escherichia coli BL21 (DE3), E. coli RV308, E. coli DH5α, E. coli JM109, E. coli XL-1, Klebsiella planticola (Rf), and among these in particular of those of the strain deposited with the application number DSM 14225.

30. The bacterial strain as claimed in any of claims 25 to 29, which expresses the recombinant protein under the control of a promoter which need not necessarily be induced from the outside, preferably of a constitutive promoter and particularly preferably of the Bacillus amyloliquefaciens β-glucanase promoter (bgl promoter).

31. A derivative of the microorganism deposited with the application number DSM 14225.

32. A microorganism, characterized in that it has been obtained after transformation with any of the vectors as claimed in any of claims 22 to 24.

33. A method for fermentation of Gram-negative bacteria producing a recombinant protein which is at least partially secreted into the medium surrounding said bacteria with the aid of a system which partially opens the outer membrane of these bacteria, characterized in that the recombinant protein is expressed under the control of a promoter from a Gram-positive organism, preferably from an organism of the genus Bacillus, which promoter does not naturally regulate the corresponding gene or a gene highly homologous to this gene.

34. The method as claimed in claim 33, characterized in that bacteria as claimed in any of claims 25 to 31 are used.

35. The method as claimed in claim 33 or 34, characterized in that the fermentation is carried out via a continuous supply strategy.

36. The method for fermentation of a bacterial strain as claimed in any of claims 33 to 35, characterized in that the protein produced is subsequently harvested from the fermentation medium.

37. The method for fermentation of a bacterial strain as claimed in any of claims 33 to 35, characterized in that the protein produced is continuously removed during the fermentation.


SEQUENCE LISTING (6 sequences)

SEQ ID NO: 1 - DNA, 27 bp, Artificial
  PCR primer
aacgaattcaacgaagaatcgctgcac

SEQ ID NO: 2 - DNA, 30 bp, Artificial
  PCR primer
tcgcggatccttaccccttttttgaacacg

SEQ ID NO: 3 - DNA, 21 bp, Artificial
  PA02 primer
tttggatccgaaaatgagagg

SEQ ID NO: 4 - DNA, 21 bp, Artificial
  PA03 primer
attgggagctcctacgatcgc

SEQ ID NO: 5 - DNA, 171 bp, Bacillus amyloliquefaciens
tcgaccgatgttccctttgaaaaggatcatgtatgatcaataaagaaagcgtgttcaaaaaaggggtaaggatccaaggatcgagttatgaggaaaagattttttgtgggaatattcgcgataaacctccttgttggatgtcaggctaactatatacctccttggaatggc

SEQ ID NO: 6 - protein, 28 aa, Bacillus amyloliquefaciens
MRKRFFVGIFAINLLVGCQANTIPPWNG